(19) United States
(12) Patent Application Publication
(10) Pub. No.: US 2005/0050650 A1
(43) Pub. Date: Mar. 10, 2005

(54) Composition comprising at least one conductive polymer and at least one direct dye, and process for the use thereof

(76) Inventors: Isabelle Rollat-Corvol, Paris (FR); Henri Samain, Bievres (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/892,310

(22) Filed: Jul. 16, 2004

Related U.S. Application Data
(60) Provisional application No. 60/492,290, filed on Aug. 5, 2003.

(30) Foreign Application Priority Data
Jul. 16, 2003 (FR) 03 08677

(51) Int. Cl. A61K 7/13

(52) U.S. Cl. 8/405

(57) ABSTRACT

The present disclosure relates to a composition comprising, in a cosmetically acceptable medium, at least one direct dye and at least one conductive polymer. The at least one conductive polymer may, for example, comprise at least one repeating unit chosen from, for instance, aniline, pyrrole, thiophene, bisthiophene, furan, para-phenylene sulfide, vinylene para-phenylene, indole, aromatic amide, aromatic hydrazide, aromatic azomethine and aromatic ester radicals. The present disclosure also relates to a process using such a composition, to give keratin fibers an optical effect.

This application claims benefit of U.S. Provisional Application No. 60/492,290, filed Aug. 5, 2003.

The present disclosure relates to a composition comprising, in a cosmetically acceptable medium, at least one conductive polymer and at least one direct dye. The present disclosure also relates to a process for treating keratin fibers, using the abovementioned composition, for example, to give keratin fibers an optical effect.

The present disclosure further relates to the field of hair dyeing, for instance, semi-permanent dyeing using direct dyes.

Direct dyes are colored coloring agents with a certain affinity for keratin fibers. Many direct dyes are chosen from nitrobenzene dyes, azo, azomethine, methine, anthraquinone, naphthoquinone, benzoquinone, phenothiazine, indigoid, xanthene, phenanthridine, phthalocyanin and triarylmethane-based dyes and natural dyes (for example henna), alone or as mixtures.

It is not uncommon, when the aim is improving the optical effects given to keratin fibers after the dyeing process, for instance sheen effects, to incorporate specific agents into the composition. For example, hydrophobic lubricating substances, such as organic oils, waxes and silicones, are frequently used to give the fibers sheen. However, the sheen effect obtained in such a manner can lack intensity and can generally give them an artificial appearance. In addition, such compositions can have the drawback of giving the fibers a greasy or tacky feel. Finally, the presence of compounds of this type may limit the uptake of the dye into the fibers and consequently give less intense colorations.

Accordingly, one aspect of the present disclosure is thus to propose compositions comprising at least one direct dye, which can give treated keratin fibers a sheen appearance without the drawbacks encountered with the standard compositions, while at the same time maintaining good coloration properties, for instance fast, strong and sparingly selective colors. Further, the fibers can beneficially have a soft, pleasant feel.

An aspect of the present disclosure is thus a composition comprising, in a cosmetically acceptable medium:
at least one direct dye, and
at least one conductive polymer.

The present disclosure also relates to a process for treating human keratin fibers, for instance the hair, with a composition comprising conductive polymers.

According to one aspect of the process, the process comprises applying a composition as defined above to wet or dry fibers, and the medium of the composition is then evaporated off or left to evaporate off at a temperature ranging from 20 to 120° C., such as from 20 to 80° C., until the fibers are dry.

According to another aspect of the process as disclosed herein, the composition according to the present disclosure is applied to wet or dry fibers. The fibers are then optionally rinsed, they are optionally washed and rinsed, and the said fibers are then dried or left to dry at a temperature ranging from 20 to 120° C., such as from 20 to 80° C.

Another aspect of the present disclosure is likewise the use of a composition comprising at least one direct dye and at least one conductive polymer, to give keratin fibers an optical effect.

For example, the composition according to the present disclosure can uniformly give the entire head of hair, for example, a sheen that is more intense, more natural and more aesthetic than within the means of the prior art.

Moreover, when the at least one conductive polymer comprised in the composition as disclosed herein absorb in the visible spectrum, an optical effect, for instance sheen, and color are obtained simultaneously. This effect may make it possible to broaden the range of colors that may be obtained, or alternatively to optimize the contents of direct dyes.

However, other characteristics of the present disclosure will emerge more clearly upon reading the description and the examples that follow.

In the text hereinbelow and unless otherwise indicated, the limits of a range of values are understood as forming part of that range.

For the purposes of the present disclosure, the term “optical effect” covers sheen, color, metallic, goniochromatic and moiré effects.

Moreover, for instance, it should be noted that the sheen corresponds to the light intensity reflected at an angle α when the lock of hair is illuminated under an angle −α. The angle α used to measure this specular reflection, i.e., sheen, is equal to 20°. This provision of sheen is measured using a glossmeter as described, for example, in ISO standard 2813-1994 from AFNOR (August 1994, amended February 1997).

Conductive polymers

According to the present disclosure, the term “conductive polymer” means a molecular structure in which the monomer(s) has (have) high electron delocalization and whose arrangement in the polymer skeleton allows the π orbitals to overlap. This chemical characteristic is reflected by electrical conduction, which may or may not be accompanied by absorption in the UV-visible spectrum, or even in the infrared spectrum.

For the purposes of the present disclosure, the expression “conductive polymer absorbing in the visible spectrum” means any conductive polymer having a non-zero absorbance in the wavelength ranging from 400 to 800 nm, even if the absorption maxima of the polymer are outside this range.

The conductive polymers used in the context of the present disclosure are conductive polymers that are soluble or dispersible in the cosmetic medium suitable for use.

The conductive polymer is said to be soluble in the medium when it forms an isotropic clear liquid at 25° C. in the medium comprising water or a water/solvent mixture, this being obtained throughout all or part of a concentration of conductive polymer ranging from 0.01% to 50% by weight.

For example, the conductive polymers that may be used in the context of the present disclosure include conductive polymers that are soluble or dispersible in an aqueous medium, such as water.

The conductive polymer is said to be dispersible in the medium comprising water or a water/solvent mixture if, at 0.01% by weight, at 25° C., it forms a stable suspension of fine, generally spherical particles. The mean size of the particles comprising the dispersion is less than 1 μm, for instance, ranging from 5 to 400 nm, such as from 10 to 250 nm. These particle sizes are measured by light scattering.

It is worth noting that these polymers do not require the use of a dispersant.

In one embodiment of the present disclosure, the conductive polymers are in a form that is soluble in the medium of the composition.

In general, the polymers may furthermore, have, for instance, a conductivity ranging from 10⁻⁵ to 5×10⁵ siemens/cm, such as from 10⁻³ to 10⁵ siemens/cm and from 10⁻¹ to 10⁴ siemens/cm.

The conductivity is measured using a current generator (RM2 Test Unit sold by the company Jandel) equipped with a four-point measuring head (Universal four-point probes sold by the company Jandel). The four points, aligned and separated by the same space (d), are applied by simple pressure to the sample to be analysed. A current (I) is injected via the outer points using the current source, thus creating a variation in potential. The voltage (U) is measured between the two inner points connected to the voltmeter of the current generator.

In this configuration, the conductivity of the sample expressed in siemens (S) per cm, or S/cm, is given by the following expression:
σ=(K×l)/(U×e)
wherein:

K is a coefficient depending on the position of the contacts on the surface of the sample; when the points are aligned and equidistant, K is equal to: π/log(2);
l: value of the injected current, expressed in amperes

U: the measured voltage value, expressed in volts

e: thickness of the sample, expressed in cm.

This expression can be used only when the thickness of the material is negligible compared with the distance (d) existing between two points (e/d<0.25). In order to obtain sufficiently small thicknesses and thus to be able to calculate the conductivity of the material, it is recommended to perform the measurement on a non-conductive support (for example a glass slide) coated with the material to be analysed, obtained by evaporation of a dilute solution. In order to improve the homogeneity of the coating to be analysed, it is also recommended to use the deposition technique known as spin coating.

According to another embodiment of the present disclosure, the at least one conductive polymer is chosen from polymers comprising at least one repeating unit of the following formulae:
anilines of formula (I): 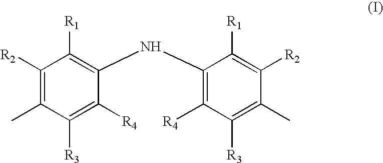
pyrroles of formulae (IIa) and (IIB): 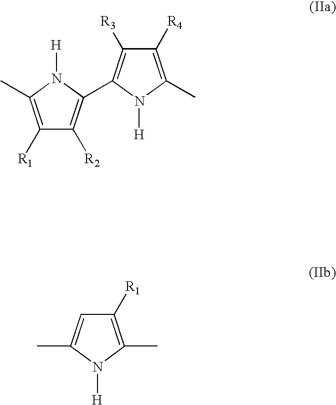
thiophenes and bisthiophenes of formulae (IIIa), (IIIb) and (IIIc): 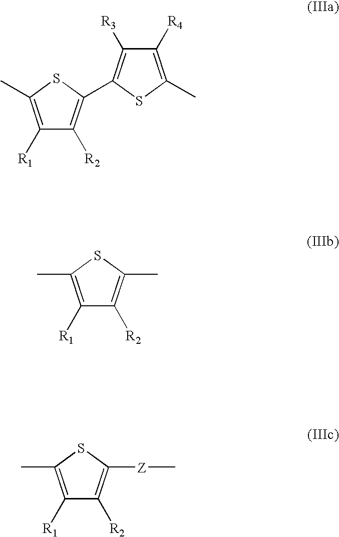
furans of formula (IV): 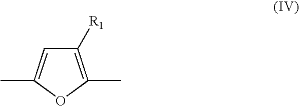
para-phenylene sulfides of structure (V) below: 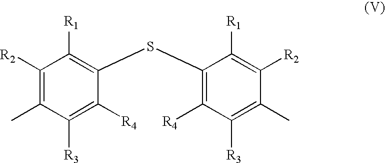
para-phenylenevinylenes of formula (VI): 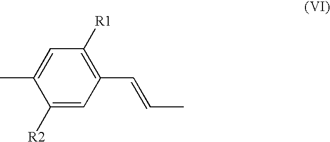
indoles of formula (VII): 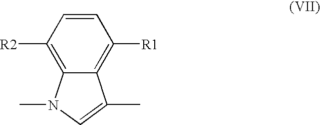
aromatic amides of formulae (VIIIa), (VIIIb), (VIIIc) and (VIIId): 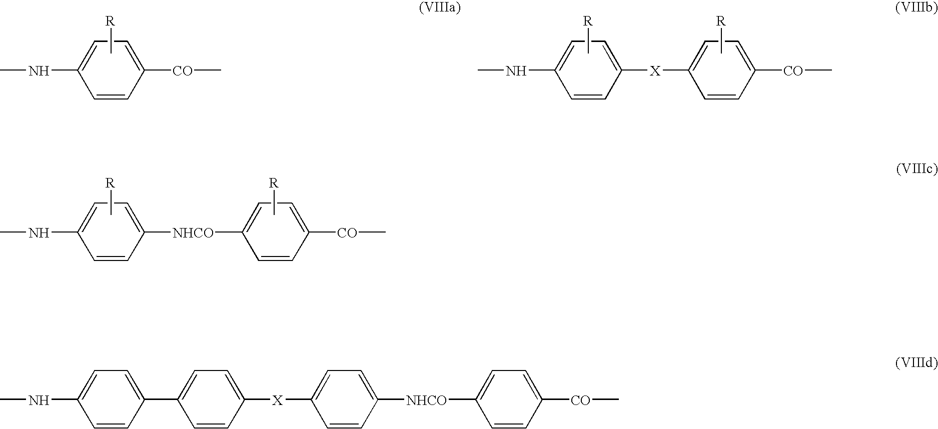
aromatic hydrazides of formulae (IXa), (IXb) and (IXc): 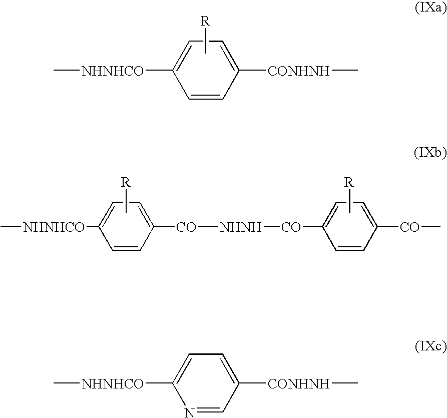
aromatic azomethines of formulae (Xa), (Xb) and (Xc): 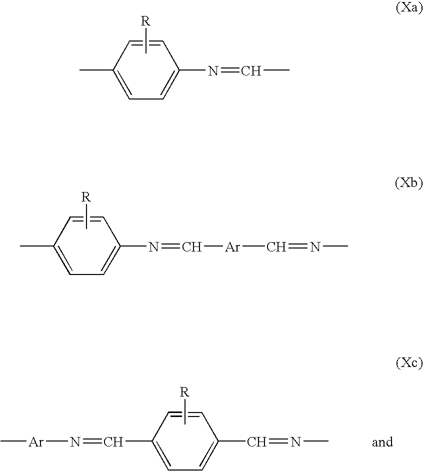
aromatic esters of formulae (XIa), (XIb) and (XIc): 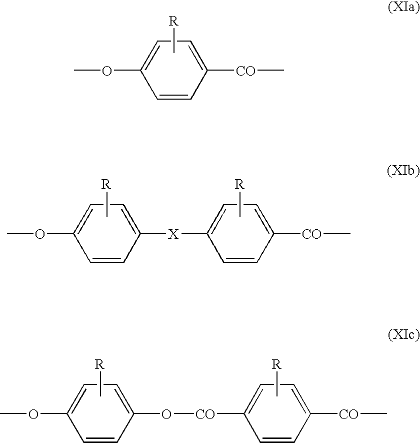
   wherein in formulae (I) to (XI):
the radicals R, R1, R2, R3, and R4, which may be identical or different, are chosen from hydrogen atoms, and -R′, —OR′, —COOR′, and —OCOR′ radicals, wherein R′ is chosen from linear and branched C₁-C₂₀ alkyl radicals, halogens, nitro radicals, cyano radicals, cyanoalkyl radicals, solubilizing groups, and solubilizing groups comprising a spacer group that bonds to the ring;
Ar is a radical comprising a radical chosen from monoaromatic and polyaromatic radicals;
X is chosen from oxygen and sulfur atoms, and —NHCO—, —SO₂—, —N═N—, —C(CH₃)₂—, —CH₂—, —CH═CH—, and —CH═N— radicals;
Z is chosen from —CH═CH— and —C≡C— radicals.

By way of non-limiting example, Ar may be at least one radical chosen from the following:

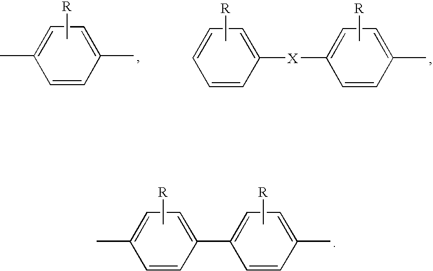

For the purposes of the present disclosure, the term “solubilizing group” means a group that ensures the dissolution of the said molecule in the cosmetic medium, such that the polymer has a conductive nature after drying the composition.

The at least one conductive polymer of the composition according to the present disclosure may comprise at least one repeating unit comprising at least one solubilizing group, and at least one other such unit lacking the groups.

Among the solubilizing groups that may be used, for example, non-limiting mention may be made of those chosen from:
carboxylic (—COOH) and carboxylate (—COO—M⁺) radicals, wherein M is chosen from alkali metals such as sodium or potassium, alkaline-earth metals, organic amines such as a primary, secondary and tertiary amines, alkanolamines and amino acids,
sulfonic (—SO₃H) and sulfonate (—SO₃⁻M⁺) radicals, wherein M has the same definition as above,
primary, secondary and tertiary amine radicals,
quaternary ammonium radicals such as —NR′₁₃⁺Z⁻, wherein Z is chosen from Br and Cl atoms, and (C₁-C₄)alkyl-OSO₃ radicals, wherein R′, which may be identical or different, are chosen from linear and branched C₁ to C₂₀ alkyl radicals, or two R′ may form a heterocycle with the nitrogen,
hydroxyl radicals, and
poly((C₂-C₃)alkylene oxide) radicals.

The carboxylic and sulfonic acid radicals may optionally be neutralized with a base, such as sodium hydroxide, 2-amino-2-methylpropanol, triethylamine and tributylamine, for example.

The amine radicals may optionally be neutralized with a mineral acid, such as hydrochloric acid, or with an organic acid, such as acetic acid or lactic acid, for example.

In addition, it is possible for the solubilizing radicals to be connected to the ring via a spacer group, for instance via -R″-, —OR″-, —OCOR″- and —COOR″- radicals, wherein R″ is chosen from linear and branched C₁-C₂₀ alkyl radicals optionally comprising at least one hetero atom, for instance oxygen.

For example, the radicals R, R1, R2, R3, and R4, which may be identical or different, are chosen from hydrogen atoms, and R′, —OR′, —OCOR′ and —COOR′ radicals, wherein R′ is chosen from linear and branched C₁-C₆ alkyl radicals; and from the optionally neutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, and —[(CH₂)₂O]ₓCH₂CH₂OCH₃ with x being an average number ranging from 0 to 200.

In one embodiment, the number n of repeating units in the polymer can range from 5 to 10,000, for instance from 5 to 1,000, such as from 10 to 1,000 and from 20 to 700.

In another embodiment of the present disclosure, the at least one conductive polymer is such that at least one radical from among R, R1, R2, R3, and R4 is a solubilizing group.

In accordance with still another embodiment of the present disclosure, the at least one conductive polymer comprises at least one solubilizing group per repeating unit.

In yet another embodiment of the present disclosure, the at least one conductive polymer is soluble in the medium of the composition.

The conductive polymers present in the composition as disclosed herein are known to those skilled in the art and are described, for example, in the book “Handbook of Organic Conductive Molecules and Polymers”—Wiley 1997—New York, Vols. 1, 2, 3; and also in the review Can. J. Chem. Vol. 64,1986.

Polythiophenes and their synthesis are further described, for example, in the article taken from the review Chem. Mater. 1998, Vol. 10, No 7, pages 1990-1999 by the authors Rasmussen S. C., Pickens J. C. and Hutchison J. E. “A new, general approach to tuning the properties of functionalized polythiophenes: The oxidative polymerization of monosubstituted bithiophenes;” in the article taken from the review Macromolecules, 1998, 31, pages 933-936, by the same authors, “Highly conjugated, water-soluble polymers via direct oxidative polymerization of monosubstituted bithiophenes.” In addition to polymerization via chemical or electrochemical oxidation, polythiophenes may also be obtained by polycondensation (dihalothiophene; catalysis with nickel or palladium complexes); via Suzuki coupling (coupling between a halogen function, for example bromine, and a boronic acid, catalysis: palladium complex and base; this then gives coupling of AA-BB type (reaction of monomers of the type A-X-A with B-X′-B) or of A-B type (reaction of several monomers of the type A-X-B); via Stille coupling (formation of a carbon-carbon bond in the presence of a Pd-based catalyst - AA-BB or A-B type); via Reike polymerization (organozinc in the presence of a nickel complex); via polymerization of McCulloch type, and so forth.

Conductive polymers that may be used in the composition according to the present disclosure are moreover described in international patent application WO 99/47570.

Among the conductive polymers that are suitable for use in the present disclosure, non-limiting mention may be made, for example, of the polymers formulae (IIIa), (IIIb) and (IIIc) in which the solubilizing groups are for instance, chosen from carboxylic acid groups; sulfonic acid groups; tertiary amine radicals; quaternary ammonium radicals such as —NR′₃⁺Z⁻ wherein Z is chosen from Br and Cl atoms, and (C₁-C₄)alkyl-OSO₃ radicals, and wherein R′, which may be identical or different, are chosen from linear and branched C₁ to C₂₀ alkyl radicals, or two R′ radicals may form a heterocycle with the nitrogen; the solubilizing groups being optionally connected to the ring via a spacer. The carboxylic and sulfonic acid functional groups may optionally be neutralized.

Thus, the polymerization may be performed via chemical or electrochemical oxidation of the corresponding thiophene monomer or else via polycondensation.

By way of non-limiting example, the polythiophenes of formulae (IIIa) and (IIIb) may be obtained by polymerization via oxidation (for example with FeCl₃ catalysis); via polycondensation of dihalothiophene catalyzed with nickel or palladium complexes (e.g.: NiCl₂(dppe)₂); via Suzuki coupling (coupling between a halogen function, for example bromine, and a boronic acid, catalysis: palladium complex and base; this then gives coupling of AA-BB type (reaction of monomers of the type A-X-A with B-X′-B) or of A-B type (reaction of several monomers of the type A-X-B); via Stille coupling (formation of a carbon-carbon bond formed in the presence of a Pd-based catalyst—AA-BB or A-B type); via Reike polymerization (organozinc in the presence of a nickel complex); via polymerization of McCulloch type, and so forth.

The vinylene polythiophenes of formula (IIIc) wherein Z is a —CH═CH—radical, may be obtained for example, via Gilch polymerization in the presence of a strong base (potassium tert-butoxide) of 2,5-bis(bromoalkylene)thiophene; via polymerization by the Wessling method via the use of a precursor based on sulfonium salts and pyrolysis; and via a Wittig-Horner Wittig reaction.

The ethynylene polythiophenes of formula (IIIc) wherein Z is a —C≡C— radical, may be obtained by Heck-Sonogashira coupling (of the type AA-BB or A-B; formation of a carbon-carbon bond between a terminal acetylenic (or true acetylenic) function and a bromo or iodo function, catalyzed with a palladium/copper complex (PdCl₂(PPh₃)₃, CuI or Cu(OAc)₂) in the presence of a base such as triethylamine, diisopropyl amine, piperidine, and so forth); and via metathesis of alkynes in the presence of a molybdenum complex (Mo(CO)₆).

In general, the functionalization of the polythiophenes, i.e., the introduction of the solubilizing or non-solubilizing group(s), is performed on the monomer before it is polymerized.

In other cases, the solubilizing group is obtained after working up the polymer. For example, this is the case for the carboxylic acid functional group, which may be obtained by hydrolysis of the corresponding ester.

Among the solubilizing groups that may be used, non-limiting mention may be made of, for instance, carboxylic acid radicals; sulfonic acid radicals; tertiary amine radicals; and quaternary ammonium radicals such as —NR′₃⁺Z⁻ wherein Z is chosen from Br and Cl atoms, (C₁-C₄)alkyl-OSO₃ radicals and the salts thereof, and wherein R′, which may be identical or different, are chosen from linear and branched C₁-C₂₀ alkyl radicals, optionally connected to the ring via a spacer, such as C₁-C₂₀ alkyl radicals. The carboxylic and sulfonic acid radicals may optionally be neutralized.

According to one embodiment of the present disclosure, for example, the at least one conductive polymer is chosen from those of formulae (IIIa), (IIIb) and (IIIc), wherein at least one radical chosen from R1, R2, R3, and R4 of formula (IIIa), and R1 and R2 of formulae (IIIb) and (IIIc) is a solubilizing group of carboxylic acid type, in neutralized or non-neutralized form, optionally connected to the ring via a spacer, for example linear and branched C₁-C₂₀ alkyl radicals, wherein the other radical(s) are hydrogen atoms.

In another embodiment, the at least one conductive polymer can be present in the composition in and amount of at least 0.001% by weight, for instance, of at least 0.01% by weight, such as of at least 0.1% by weight and of at least 0.5% by weight, relative to the total weight of the composition. Moreover, the amount of the at least one conductive polymer can be, for example, up to 50% by weight, for instance, up to 30% by weight, such as up to 20% by weight and up to 10% by weight, relative to the total weight of the composition.

According to another embodiment of the present disclosure, for instance, the amount of the at least one conductive polymer ranges from 0.1% to 50% by weight, such as from 0.1% to 30% by weight and from 0.5% to 10% by weight, relative to the total weight of the composition.

Direct dyes

For example, the at least one direct dye is chosen from nonionic, cationic and anionic nature. In general, these direct dyes may be chosen from nitrobenzene dyes, azo, azomethine, methine, anthraquinone, naphthoquinone, benzoquinone, phenothiazine, indigoid, xanthene, phenanthridene, phthalocyanin and triarylmethane-based dyes, alone or as mixtures.

Among the direct dyes that may be used, non-limiting mention may be made of those chosen, for example, from the following red or orange nitrobenzene dyes:
1-hydroxy-3-nitro-4-N-(γ-hydroxypropyl)aminobenzene,
N-(β-hydroxyethyl)amino-3-nitro-4-aminobenzene,
1-amino-3-methyl-4-N-(β-hydroxyethyl)amino-6-nitrobenzene,
1-hydroxy-3-nitro-4-N-(β-hydroxyethyl)aminobenzene,
1,4-diamino-2-nitrobenzene,
1-amino-2-nitro-4-methylaminobenzene,
N-(β-hydroxyethyl)-2-nitroparaphenylenediamine,
1-amino-2-nitro-4-(β-hydroxyethyl)amino-5-chlorobenzene,
2-nitro-4-aminodiphenylamine,
1-amino-3-nitro-6-hydroxybenzene.
1-(β-aminoethyl)amino-2-nitro-4-(β-hydroxyethyloxy)benzene,
1-(β,γ-dihydroxypropyl)oxy-3-nitro-4-(β-hydroxyethyl)aminobenzene,
1-hydroxy-3-nitro-4-aminobenzene,
1-hydroxy-2-amino-4,6-dinitrobenzene,
1-methoxy-3-nitro-4-(β-hydroxyethyl)aminobenzene,
2-nitro-4′-hydroxydiphenylamine, and
1-amino-2-nitro-4-hydroxy-5-methylbenzene.

The composition according to the present disclosure may also comprise in addition to or in replacement of the above nitrobenzene dyes, at least one direct dye chosen from yellow, green-yellow, blue or violet nitrobenzene dyes, azo dyes, anthraquinone, naphthoquinone, benzoquinone, indigoid dyes and triarylmethane-based dyes.

The at least one direct dye may be, for example, basic dyes, among which non-limiting mention may be made, for instance, of the dyes known in the Color Index, 3rd edition, under the names “Basic Brown 16”, “Basic Brown 17”, “Basic Yellow 57”, “Basic Red 76”, “Basic Violet 10”, “Basic Blue 26” and “Basic Blue 99”; and acidic direct dyes, among which non-limiting mention may be made, for example, of the dyes known in the Color Index, 3rd edition, under the names “Acid Orange 7”, “Acid Orange 24”, “Acid Yellow 36”, “Acid Red 33”, “Acid Red 184”, “Acid Black 2”, “Acid Violet 43”, and “Acid Blue 62”, and cationic direct dyes such as those described in patent application numbers WO 95/01772, WO 95/15144 and EP 714 954, the content of which forms an integral part of the present invention.

Among the additional yellow and green-yellow nitrobenzene direct dyes that may be used as disclosed herein, non-limiting mention may be made, for example, of the compounds:
1-β-hydroxyethyloxy-3-methylamino-4-nitrobenzene,
1-methylamino-2-nitro-5-(β,γ-dihydroxypropyl)oxybenzene,
1-(β-hydroxyethyl)amino-2-methoxy-4-nitrobenzene,
1-(β-aminoethyl)amino-2-nitro-5-methoxybenzene,
1,3-di(β-hydroxyethyl)amino-4-nitro-6-chlorobenzene,
1-amino-2-nitro-6-methylbenzene,
1-(β-hydroxyethyl)amino-2-hydroxy-4-nitrobenzene,
N-(β-hydroxyethyl)-2-nitro-4-trifluoromethylaniline,
4-(β-hydroxyethyl)amino-3-nitrobenzenesulfonic acid,
4-ethylamino-3-nitrobenzoic acid,
4-(β-hydroxyethyl)amino-3-nitrochlorobenzene,
4-(β-hydroxyethyl)amino-3-nitromthylbenzene,
4-(β,γ-dihydroxypropyl)amino-3-nitrotrifluoromethylbenzene,
1-(β-ureidoethyl)amino-4-nitrobenzene,
1,3-diamino-4-nitrobenzene,
1-hydroxy-2-amino-5-nitrobenzene,
1-amino-2-[tris(hydroxymethyl)methyl]amino-5-nitrobenzene,
1-(β-hydroxyethyl)amino-2-nitrobenzene,
4-(β-hydroxyethyl)amino-3-nitrobenzamide.

Among the additional blue or violet nitrobenzene direct dyes that may be used according to the present disclosure, non-limiting mention may be made, for example, of the compounds chosen from:
1-(β-hydroxyethyl)amino-4-N,N-bis(β-hydroxyethyl)amino-2-nitrobenzene,
1-(γ-hydroxypropyl)amino-4-N,N-bis(β-hydroxyethyl)amino-2-nitrobenzene,
1-(β-hydroxyethyl)amino-4-(N-methyl-N-β-hydroxyethyl)amino-2-nitrobenzene,
1-(β-hydroxyethyl)amino-4-(N-ethyl-N-β-hydroxyethyl)amino-2-nitrobenzene,
1-(β,γ-dihydroxypropyl)amino-4-(N-ethyl-N-β-hydroxyethyl)amino-2-nitrobenzene,
the 2-nitro-para-phenylenediamines of the following formula: 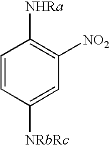
   wherein:
Rb is chosen from C₁-C₄ alkyl radicals and β-hydroxyethyl, β-hydroxypropyl and γ-hydroxypropyl radicals;
Ra and Rc, which may be identical or different, are chosen from β-hydroxyethyl, β-hydroxypropyl, γ-hydroxypropyl and β,γ-dihydroxypropyl radicals, wherein at least one of the radicals Rb, Rc and Ra is a γ-hydroxypropyl radical, and with the proviso that Rb and Rc are not simultaneously a β-hydroxyethyl radical when Rb is a γ-hydroxypropyl radical, such as those described in French Patent No. FR 2,692,572.

In one embodiment, the at least one direct dye is present in the composition in an amount ranging from 0.0005% to 12% by weight, relative to the total weight of the composition, for instance, from 0.005% to 6% by weight, relative to the total weight of the composition.

Surfactants

For example, the cosmetic composition according to the present disclosure comprises at least one surfactant, which may be chosen, without preference, from anionic, amphoteric, nonionic, zwitterionic, cationic surfactants, and mixtures thereof.

Among the surfactants that are suitable for use according to the present disclosure, for example, non-limiting mention may be made of the following:

(i) Anionic surfactant(s):

By way of non-limiting example of anionic surfactants that can be used, alone or as mixtures, in the context of the present disclosure, non-limiting mention may be made for instance of salts, such as alkali metal salts, sodium salts, ammonium salts, amine salts, amino alcohol salts and magnesium salts, of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; (C₆-C₂₄)alkyl sulfosuccinates, (C₆-C₂₄)alkyl ether sulfosuccinates, (C₆-C₂₄)alkylamide sulfosuccinates; (C₆-C₂₄)alkyl sulfoacetates; (C₆-C₂₄)acyl sarcosinates; and (C₆-C₂₄)acyl glutamates. It is also possible to use (C₆-C₂₄)alkylpolyglycoside carboxylic esters such as alkylglucoside citrates, alkylpolyglycoside tartrate and alkylpolyglycoside sulfosuccinates, alkylsulfosuccinamates; acyl isethionates and N-acyl taurates; the alkyl or acyl radical of all of these different compounds, for example, comprising from 12 to 20 carbon atoms and the aryl radical for instance denoting a phenyl or benzyl group. Among the anionic surfactants which can also be used, non-limiting mention may also be made of fatty acid salts such as oleic, ricinoleic, palmitic and stearic acid salts, coconut oil acid or hydrogenated coconut oil acid; acyl lactylates in which the acyl radical comprises 8 to 20 carbon atoms. It is also possible to use alkyl D-galactoside uronic acids and their salts, polyoxyalkylenated (C₆-C₂₄)alkyl ether carboxylic acids, polyoxyalkylenated (C₆-C₂₄)alkylaryl ether carboxylic acids, polyoxyalkylenated (C₆-C₂₄)alkylamido ether carboxylic acids and their salts, for example, those comprising from 2 to 50 alkylene oxide groups, such as ethylene oxide groups, and mixtures thereof.

(ii) Nonionic surfactant(s):

The nonionic surfactants are, themselves also, compounds that are well known per se (see for example, in this respect “Handbook of Surfactants” by M. R. Porter, published by Blackie & Son (Glasgow and London), 1991, pp. 116-178) and their nature is not a critical factor in the context of the present invention. Thus, non-limiting mention can be of polyethoxylated or polypropoxylated, alkylphenols, alpha-diols or alcohols, having a fatty chain comprising, for example, 8 to 18 carbon atoms, it being possible for the number of ethylene oxide or propylene oxide groups to range, for example, from 2 to 50. Non-limiting mention may also be made of copolymers of ethylene oxide and of propylene oxide, condensates of ethylene oxide and of propylene oxide with fatty alcohols; polyethoxylated fatty amides having for instance, from 2 to 30 mol of ethylene oxide, polyglycerolated fatty amides comprising on average 1 to 5, and such as 1.5 to 4, glycerol groups; polyethoxylated fatty amines having for example, from 2 to 30 mol of ethylene oxide; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, and amine oxides such as (C₁₀-C₁₄)alkylamine oxides or N-acylaminopropylmorpholine oxides.

(iii) Amphoteric or zwitterionic surfactant(s):

The amphoteric or zwitterionic surfactants can be chosen for example, from aliphatic secondary and tertiary amine derivatives wherein the aliphatic radical is a linear or branched chain comprising 8 to 18 carbon atoms and comprising at least one water-solubilizing anionic group (for example carboxylate, sulfonate, sulfate, phosphate or phosphonate); non-limiting mention may also be made of (C₈-C₂₀)alkylbetaines, sulfobetaines, (C₈-C₂₀)alkylamido(C₁-C₆)alkylbetaines or (C₈-C₂₀)alkylamido(C₁-C₆)alkylsulfobetaines.

Among the amine derivatives, non-limiting mention may be made of the products sold under the name Miranol, as described in U.S. Pat. Nos. 2,528,378 and 2,781,354 and classified in the CTFA dictionary, 3rd edition, 1982, under the names Amphocarboxyglycinates and Amphocarboxypropionates, with the respective structures:
R_{d}—CONHCH₂CH₂—N(Rₑ)(R_{f})(CH₂COO⁻)
wherein: R_{d} is chosen from alkyl radicals of an acid R_{d}—COOH present in hydrolysed coconut oil, and heptyl, nonyl and undecyl radicals, Rₑ is a beta-hydroxyethyl group and R_{f} is a carboxymethyl group; and
R_{g}—CONHCH₂CH₂—N(B)(C)
wherein:

B is —CH₂CH₂OX, C is —(CH₂)_{z}—Y, wherein z is equal to 1 or 2,

X is a —CH₂CH₂—COOH group or a hydrogen atom,

Y is —COOH or a —CH₂—CHOH—SO₃H radical,

R_{g} is chosen from alkyl radicals of an acid Rₕ—COOH present in coconut oil or in hydrolysed linseed oil, a saturated radical or a radical comprising at least one unsaturation, for example of C₇ to C₁₇, such as a C₉, C₁₁, C₁₃ or C₁₇ alkyl radical or its iso form, and an unsaturated C₁₇ radical.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Caprylamphodipropionate, Lauroamphodipropionic acid and Cocoamphodipropionic acid.

By way of example, non-limiting mention may be made of the cocoamphodiacetate sold under the trade name Miranol® C2M concentrate by the company Rhodia Chimie.

(iv) Cationic surfactants:

Among the cationic surfactants, non-limiting mention may be made for example of salts of optionally polyoxyalkylenated primary, secondary and tertiary fatty amines; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives; or amine oxides of cationic nature.

In one embodiment, the amount of surfactants present in the composition according to the invention can range from 0.01% to 40% by weight, for instance from 0.5% to 30% by weight, relative to the total weight of the composition.

Medium

The cosmetically acceptable medium of the composition can be, for example, an aqueous medium comprising water and also, for example comprising at least one cosmetically acceptable organic solvent including, for instance, C₁-C₄ alcohols such as ethyl alcohol, isopropyl alcohol, aromatic alcohols such as benzyl alcohol and phenylethyl alcohol, or glycols or glycol ethers such as, for example, ethylene glycol monomethyl, monoethyl or monobutyl ether, propylene glycol or ethers thereof such as, for example, propylene glycol monomethyl ether, butylene glycol, dipropylene glycol and also diethylene glycol alkyl ethers, for instance diethylene glycol monoethyl ether or monobutyl ether, or alternatively polyols, for instance glycerol. Polyethylene glycols and polypropylene glycol and mixtures of all these compounds may also be used as solvent.

The organic solvents may, for example, be present in an amount ranging from 0.5% to 20%, and such as ranging from 2% to 10% by weight, relative to the total weight of the composition.

Additives

The cosmetic composition may also comprise an effective amount of at least one other agent, which are previously known elsewhere in the dyeing of human keratin fibers, such as thickeners, antioxidants, fragrances, dispersants, conditioners for instance, including cationic or amphoteric polymers, opacifiers, sequestering agents such as EDTA and etidronic acid, UV-screening agents, waxes, volatile or non-volatile, cyclic or linear or branched silicones, which are organomodified (such as by amine groups) or unmodified, preserving agents, ceramides, pseudoceramides, plant, mineral or synthetic oils, vitamins or provitamins, for instance panthenol, and nonionic, anionic, amphoteric or cationic associative polymers.

Needless to say, a person skilled in the art will take care to select the optional additional compound(s) mentioned above such that the beneficial properties intrinsically associated with the dye composition according to the present disclosure are not, or are not substantially, adversely affected by the envisaged addition(s).

The composition may likewise further comprise at least one oxidizing agent.

The at least one optional oxidizing agent is chosen, for example, from hydrogen peroxide, urea peroxide, alkali metal bromates, persalts such as perborates and persulfates, and enzymes such as the peroxidases and two- or four-electron oxidoreductases. In one embodiment of the present disclosure, the at least one oxidizing agent is chosen from hydrogen peroxide and enzymes, for example.

If it is present, the at least one oxidizing agent may be present, for example, in an amount ranging from 0.001% to 10% by weight, relative to the weight of the composition.

The composition as disclosed herein is generally applied to wet or dry keratin fibers. The medium of the composition is then evaporated off, or is left to evaporate off, at a temperature ranging from 20° C. to 120° C., such as from 20° C. to 80° C., until the fibers are dry.

According to another aspect of the present disclosure, the composition is applied to wet or dry fibers and then optionally rinsing the fibers, optionally washing them, for instance with a shampoo, rinsing them, and then drying them or leaving them to dry at a temperature ranging from 20° C. to 120° C. It should be noted that, in the case of this embodiment, the leave-in time of the composition ranges from 5 to 60 minutes, for instance, from 5 to 40 minutes. For example, in the case where at least one oxidizing agent is present, this embodiment may also be performed.

The temperature at which the composition is applied can range from 15° C. to 80° C., such as from 15° C. to 40° C.

When at least one oxidizing agent is desired, the composition according to the present disclosure and the at least one oxidizing agent may be applied sequentially in any order, or even simultaneously. If simultaneous application is chosen, the composition and the at least one oxidizing agent can be for example, mixed together just before application.

It should be noted that, in accordance with still another embodiment, it is possible to store separately the composition according to the present disclosure, on the one hand, and an oxidizing composition, on the other hand. This oxidizing composition for instance, comprises at least one oxidizing agent in a suitable dyeing medium. The composition according to the present disclosure and the oxidizing composition are then mixed together at the time of use, after which this mixture is applied to the keratin fibers; and then optionally followed by rinsing the fibers, optionally washing them, for instance with a shampoo, rinsing them, and then drying them or leaving them to dry at a temperature ranging from 20° C. to 120° C.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The example that follows illustrates the present disclosure without, however, being limiting in nature.

### EXAMPLE

Synthesis of Poly(Thiophene-3-Acetic Acid)

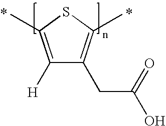

Procedure

Preparation of the polymer: poly(ethyl thiophene-3-acetate)

25 ml of dry chloroform were introduced into a Schlenk tube under argon, the system is degassed and the following reagents were then introduced: 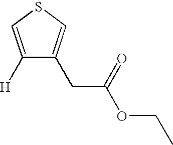
2.5 g of ethyl thiophene-3-acetate (14.7 mmol) and 1 g of FeCl₃ (6.15 mmol).

The mixture was stirred for 24 hours under argon at 50° C.

The poly(ethyl thiophene-3-acetate) polymer was then precipitated in heptane.

The polymer was then dissolved in a tetrahydrofuran solution.

Infrared characterization:

C═O band: 1719 cm⁻¹; CH₂, CH₃ bands=2979 cm⁻¹, 2934 cm⁻¹ and disappearance of the CH band at 3102 cm⁻¹ present in the monomer.

Hydrolysis of the polymer: poly(ethyl thiophene-3-acetate) to form poly(thiophene-3-acetic acid)

The polymer obtained above was then hydrolyzed with an excess of 50 ml of an aqueous sodium hydroxide solution (2N) for 48 hours at 70° C., followed by acidification with concentrated HCl up to the point of precipitation of the product: poly(thiophene-3-acetic acid).

The polymer was then filtered off and washed several times with distilled water in order to remove the traces of catalyst.

Infrared characterization of the polymer:

C═O band: 1740 cm⁻¹; COO 1580 cm⁻¹; OH (broad band 3000-3500 cm⁻¹)

Neutralization of the poly(thiophene-3-acetic acid) polymer:

The poly(thiophene-3-acetic acid) polymer (2 g) was dissolved in tetrahydrofuran (30 g) and neutralized with a proportion of 1 mol of sodium hydroxide per mole of carboxylic acid.

Water (30 g) was then added.

The tetrahydrofuran was evaporated off.

An aqueous 6% solution of poly(thiophene-3-acetic acid) in the form of a sodium salt was thus obtained.

Formulation comprising the polymer obtained: Composition:

| | |
|---|---|
| Poly(thiophene-3-acetic acid) obtained above | 5 g |
| para-Aminoazo-N,N′-dimethyl-imidazolium chloride (from Ciba) | 0.10 g |
| Aminomethyl propanol qs | pH 7 |
| Ethyl alcohol | 15 g |
| Water qs | 100 g |

The composition described above, comprising the conductive polymer obtained according to the preceding example (0.1 g per g of hair) was applied to locks of natural chestnut-brown hair at room temperature (20° C.). After a leave-in time of 20 minutes, the lock thus treated was left to dry.

The locks were colored orange-red and have a very shiny appearance with a golden glint that was especially perceptible when the locks were observed at an oblique angle. To the feel, the hair had the advantage of not seeming greasy, and the optical properties described above were maintained over time (unchanged after 12 hours).

1. A composition comprising, in a cosmetically acceptable medium:
at least one direct dye, and
at least one conductive polymer.

2. The composition according to claim 1, wherein the at least one conductive polymer comprises at least one repeating unit of the following formulae:
anilines of formula (I): 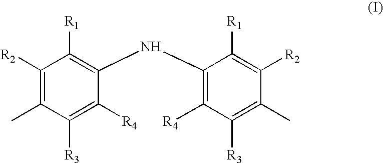
pyrroles of formulae (IIa) and (IIb): 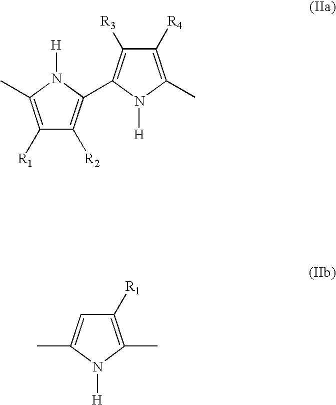
thiophenes or bisthiophenes of formulae (IIIa), (IIIb) and (IIIc): 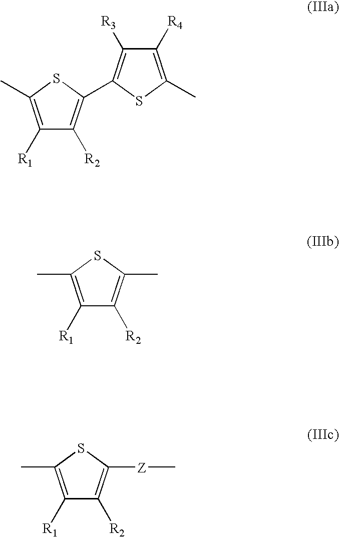
furans of formula (VI): 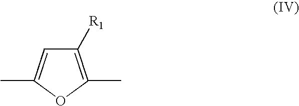
para-phenylene sulfides of formula (V): 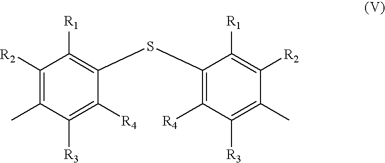
para-phenylenevinylenes of formula (VI): 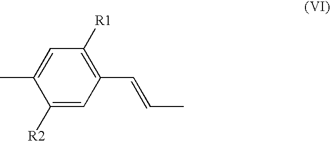
indoles of formula (VII): 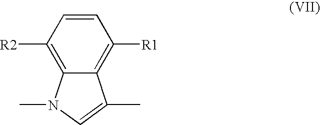
aromatic amides of formulae (VIIIa), (VIIIb), (VIIIc) and (VIIId): 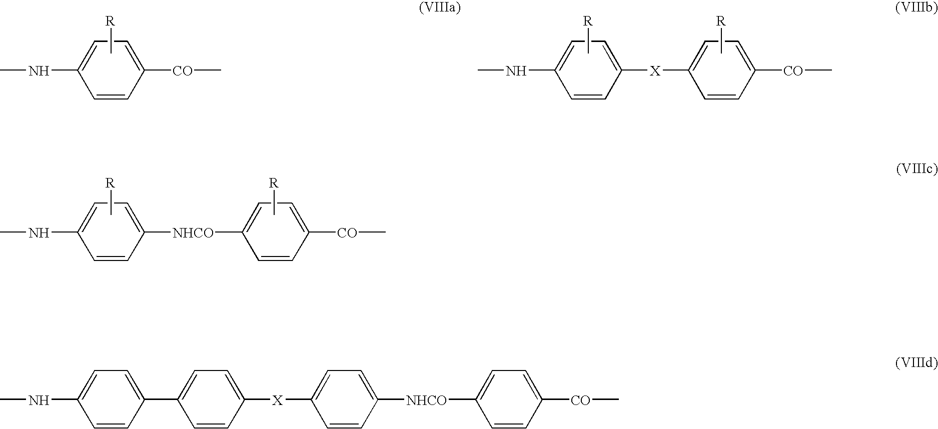
aromatic hydrazides of formulae (IXa), (IXb) and (IXc): 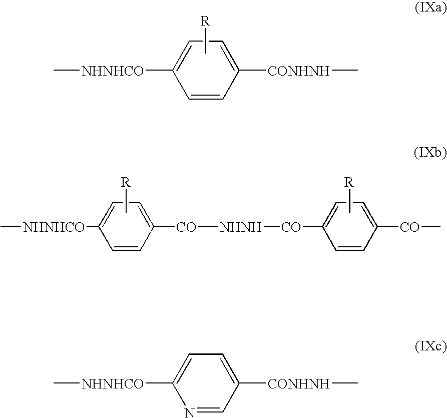
aromatic azomethines of formulae (Xa), (Xb) and (Xc): 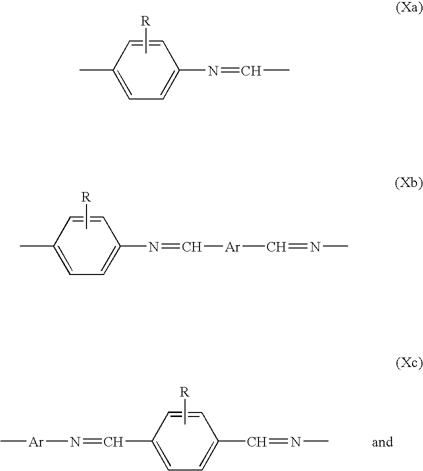
aromatic esters of formulae (XIa), (XIb) and (XIc): 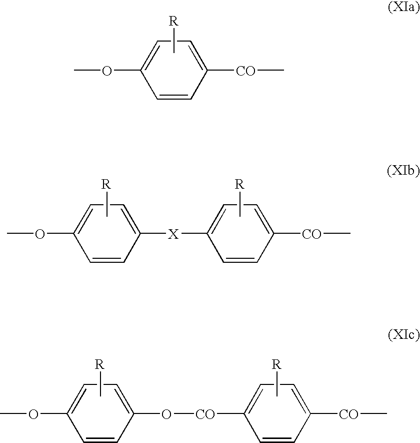
wherein in formulae (I) to (XI):
the radicals R, R1, R2, R3, and R4, which may be identical or different, are chosen from hydrogen atoms, and -R′, —OR′, —COOR′, and —OCOR′ radicals, wherein R′ is chosen from linear and branched C₁-C₂₀ alkyl radicals, halogens, nitro radicals, cyano radicals, cyanoalkyl radicals, solubilizing groups and solubilizing groups comprising a spacer group that bonds to the ring;
Ar is a radical comprising a monoaromatic or polyaromatic radical;
X is chosen from oxygen and sulfur atoms, and —NHCO—, —SO₂—, —N═N—, —C(CH₃)₂—, —CH₂—, —CH═CH—, and —CH═N— radicals; and
Z is chosen from —CH═CH— and —C≡C— radicals.

3. The composition according to claim 2, wherein the solubilizing groups are chosen from:
—COOH and —COO-M⁺, wherein M is chosen from alkali metals, alkaline-earth metals, organic amines, alkanolamines and amino acids,
—SO₃H and —SO₃⁻M⁺, wherein M is chosen from alkali metals, alkaline-earth metals, organic amines, alkanolamines and amino acids,
primary, secondary and tertiary amine radicals,
quaternary ammonium radicals,
hydroxyl radicals, and
poly((C₂-C₃)alkylene oxide) radicals.

4. The composition according to claim 2, wherein the solubilizing groups are connected to the ring via a spacer group.

5. The composition according to claim 4, wherein the spacer group is chosen from -R″-, —OR″-, —OCOR″- and —COOR″- radicals, wherein R″ is chosen from linear and branched C₁-C₂₀ alkyl radicals optionally comprising at least one hetero atom.

6. The composition according to claim 2, wherein the quaternary ammonium radicals are —NR′₃⁺Z⁻ radicals, wherein Z is chosen from Br and Cl atoms, and (C₁-C₄)alkyl-OSO₃ radicals, and wherein R′, which may be identical or different, are chosen from linear and branched C₁ to C₂₀ alkyl radicals, or two R′ radicals form a heterocycle with the nitrogen.

7. The composition according to claim 2, wherein the radicals R, R1, R2, R3 and R4, which may be identical or different, are chosen from hydrogen atoms, and R′, —OR′, —OCOR′ and —COOR′ radicals, wherein R′ is chosen from linear and branched C₁-C₆ alkyl radicals, and from the following neutralized or non-neutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, and —[(CH₂)₂O]ₓCH₂CH₂OCH₃ with x being an average number ranging from 0 to 200.

8. The composition according to claim 7, wherein at least one radical chosen from R, R1, R2, R3, and R4 of the at least one conductive polymer is a solubilizing group.

9. The composition according to claim 2, wherein the at least one conductive polymer comprises at least one solubilizing group per repeating unit.

10. The composition according to claim 3, wherein the solubilizing groups are chosen from carboxylic acid radicals; sulfonic acid radicals; tertiary amine radicals; quaternary ammonium radicals, and also the salts thereof; optionally connected to the ring via a spacer; and wherein the carboxylic acid and sulfonic acid radicals may optionally be neutralized.

11. The composition according to claim 10, wherein the quaternary ammonium radicals are —NR′₃⁺Z⁻ radicals wherein Z is chosen from Br and Cl atoms, and (C₁-C₄)alkyl-OSO₃ radicals, and wherein R′, which may be identical or different, are chosen from linear and branched C₁-C₂₀ alkyl radicals.

12. The composition according to claim 10 wherein the solubilizing groups are optionally connected to the ring via a spacer chosen from C₁-C₂₀ alkyl radicals.

13. The composition according to claim 2, wherein the at least one conductive polymer is chosen from those of formulae (IIIa), (IIIb) and (IIIc), wherein at least one radical chosen from R1, R2, R3, and R4 of formula (IIIa), and R1 and R2 of formulae (IIIb) and (IIIc) is a solubilizing group of carboxylic acid type, in neutralized or non-neutralized form, optionally connected to the ring via a spacer, and wherein the other radicals are hydrogen atoms.

14. The composition according to claim 13, wherein the spacer is chosen from linear and branched C₁-C₂₀ alkyl radicals.

15. The composition according to claim 1, wherein the at least one conductive polymer is present in an amount of at least 0.001% by weight, relative to the total weight of the composition.

16. The composition according to claim 1, wherein the at least one conductive polymer is present in an amount of up to 50% by weight, relative to the total weight of the composition.

17. The composition according to claim 1, wherein the amount of conductive polymer ranges from 0.1% to 50% by weight, relative to the total weight of the composition.

18. The composition according to claim 1, wherein the at least one conductive polymer has a conductivity ranging from 10⁻⁵ to 5×10⁻⁵ siemens/cm.

19. The composition according to claim 1, wherein the at least one direct dye is chosen from nonionic, cationic and anionic direct dyes.

20. The composition according to claim 19, wherein the at least one direct dye is chosen from nitrobenzene dyes, azo, azomethine, methine, anthraquinone, naphthoquinone, benzoquinone, phenothiazine, indigoid, xanthene, phenanthridine, phthalocyanin, triarylmethane-based dyes, and mixtures thereof.

21. The composition according to claim 1, wherein the at least one direct dye is present in an amount ranging from 0.0005% to 12% by weight, relative to the weight of the composition.

22. The composition according to claim 1, wherein the cosmetically acceptable medium is water or a mixture of water with at least one organic solvent.

23. The composition according to claim 21, wherein the at least one organic solvent is chosen from C₁-C₄ alcohols, aromatic alcohols, glycols, glycol ethers, polyols, and mixtures thereof.

24. The composition according to claim 1, further comprising at least one surfactant chosen from nonionic, anionic, cationic, amphoteric and zwitterionic surfactants.

25. A process for treating human keratin fiberscomprising:
applying to wet or dry fibers a composition comprising, in a cosmetically acceptable medium, at least one direct dye, and at least one conductive polymer;
evaporating the medium of the composition off, or leaving the medium to evaporate off at a temperature ranging from 20° C. to 120° C., until the fibers are dry.

26. The process according to claim 25, wherein the human keratin fibers are hair.

27. The process according to claim 25, wherein the medium of the composition is evaporated off at a temperature ranging from 20° C. to 80° C., until the fibers are dry.

28. The process according to 25, wherein the at least one conductive polymer comprises at least one repeating unit of the following formulae:
anilines of formula (I): 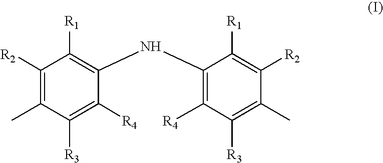
pyrroles of formulae (IIa) and (IIb): 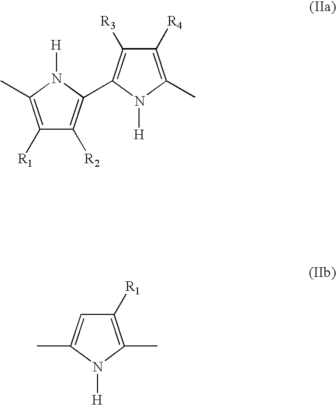
thiophenes or bisthiophenes of formulae (IIIa), (IIIb) and (IIIc): 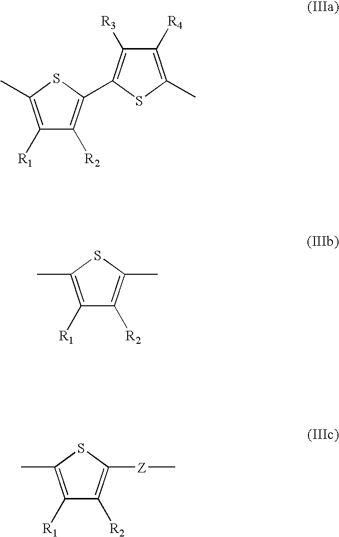
furans of formula (IV): 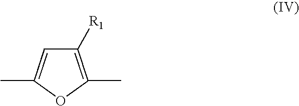
para-phenylene sulfides of formula (V): 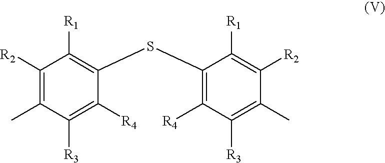
para-phenylenevinylenes of formula (VI): 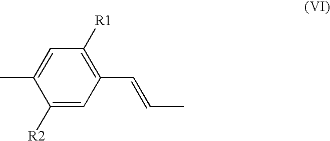
indoles of formula (VII): 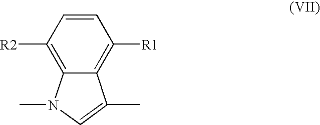
aromatic amides of formulae (VIIIa), (VIIIb) (VIIIc) and (VIIId): 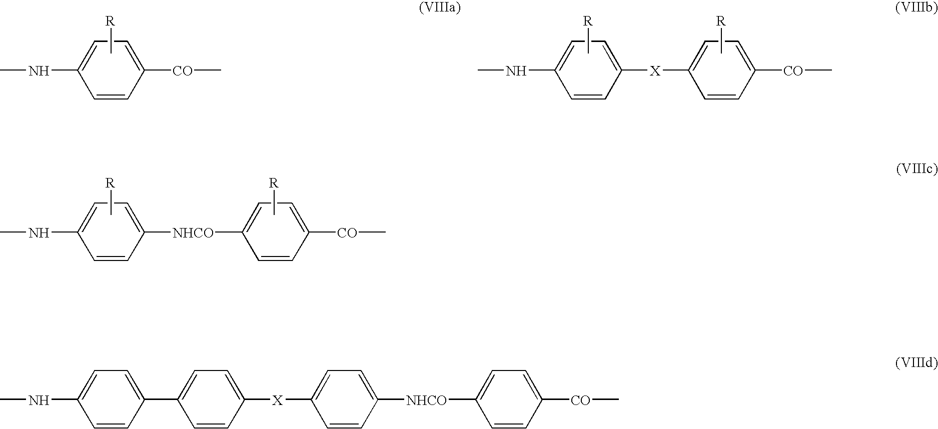
aromatic hydrazides of formulae (IXa), (IXb) and (IXc): 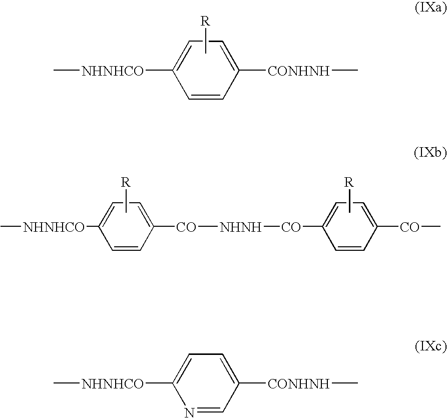
aromatic azomethines of formulae (Xa), (Xb) and (Xc): 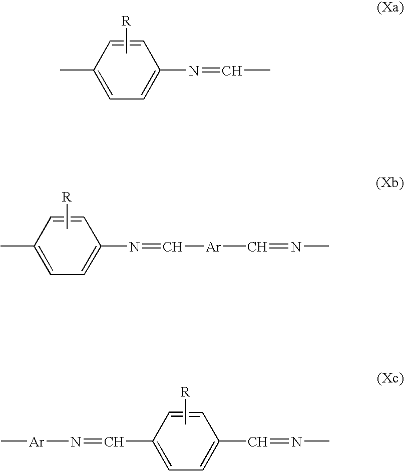
aromatic esters of formulae (XIa), (XIb) and (XIc): 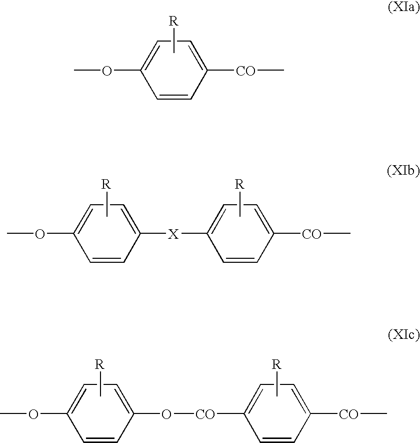
wherein in formulae (I) to (XI):
the radicals R, R1, R2, R3, and R4, which may be identical or different, are chosen from hydrogen atoms, and —R′, —OR′, —COOR′, and —OCOR′ radicals, wherein R′ is chosen from linear and branched C₁-C₂₀ alkyl radicals, halogens, nitro radicals, cyano radicals, cyanoalkyl radicals, solubilizing groups and solubilizing groups comprising a spacer group that bonds to the ring;
Ar is a radical comprising a monoaromatic or polyaromatic radical;
X is chosen from oxygen and sulfur atoms, and —NHCO—, —SO₂—, —N═N—, —C(CH₃)₂—, —CH₂—, —CH═CH—, and —CH═N— radicals; and
Z is chosen from —CH═CH— and —C≡C— radicals.

29. The process according to claim 28, wherein the solubilizing groups are chosen from:
—COOH and —COO—M⁺, wherein M is chosen from alkali metals, alkaline-earth metals, organic amines, alkanolamines and amino acids,
—SO₃H and —SO₃⁻M⁺, wherein M is chosen from alkali metals, alkaline-earth metals, organic amines, alkanolamines and amino acids,
primary, secondary and tertiary amine radicals,
quaternary ammonium radicals,
hydroxyl radicals, and
poly((C₂-C₃)alkylene oxide) radicals.

30. The process according to claim 28, wherein the solubilizing groups are connected to the ring via a spacer group.

31. The process according to claim 30, wherein the spacer group is chosen from -R″-, —OR″-, —OCOR″- and —COOR″- radicals, wherein R″ is chosen from linear and branched C₁-C₂₀ alkyl radicals optionally comprising at least one hetero atom.

32. The process according to claim 29, wherein the quaternary ammonium radicals are —NR′₃⁺Z⁻ radicals wherein Z is chosen from Br and Cl atoms, and (C₁-C₄)alkyl-OSO₃ radicals, and wherein R′, which may be identical or different, are chosen from linear and branched C₁ to C₂₀ alkyl radicals, or two R′ radicals form a heterocycle with the nitrogen.

33. A process for treating human keratin fibers comprising:
applying to wet or dry fibers a composition comprising, in a cosmetically acceptable medium, at least one direct dye, and at least one conductive polymer;
optionally rinsing the fibers,
optionally washing and rinsing the fibers,
drying the fibers or leaving them to dry at a temperature ranging from 20° C. to 120° C.

34. The process according to claim 33, wherein the human keratin fibers are hair.

35. The process according to claim 33, wherein the fibers are dried or left to dry at a temperature ranging from 20° C. to 80° C.

36. The process according to claim 33, wherein the at least one conductive polymer comprises at least one repeating unit of the following formulae:
anilines of formula (I): 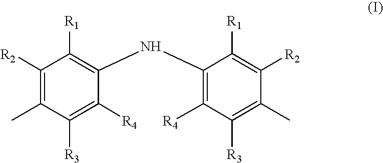
pyrroles of formulae (IIa) and (IIb): 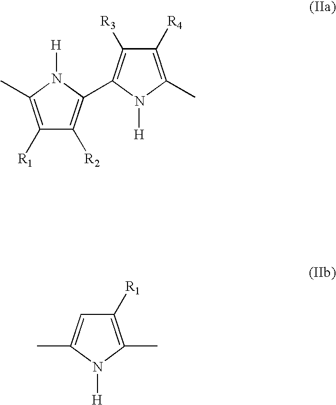
thiophenes or bisthiophenes of formulae (IIIa), (IIIb) and (IIIc): 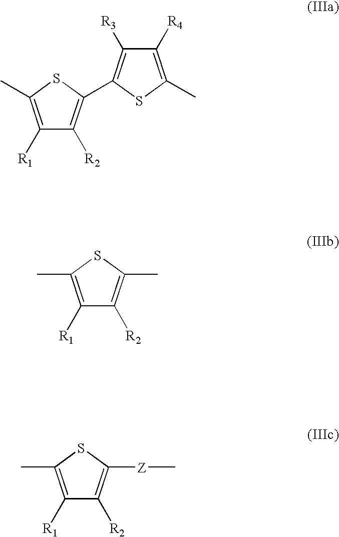
furans of formula (IV): 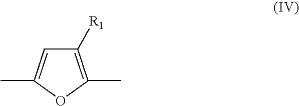
para-phenylene sulfides of formula (V): 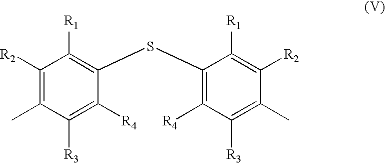
para-phenylenevinylenes of formula (VI): 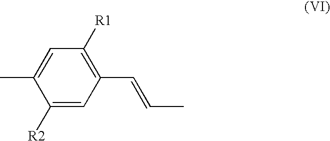
indoles of formula (VII): 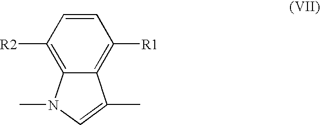
aromatic amides of formulae (VIIIa), (VIIIb), (VIIIc) and (VIIId): 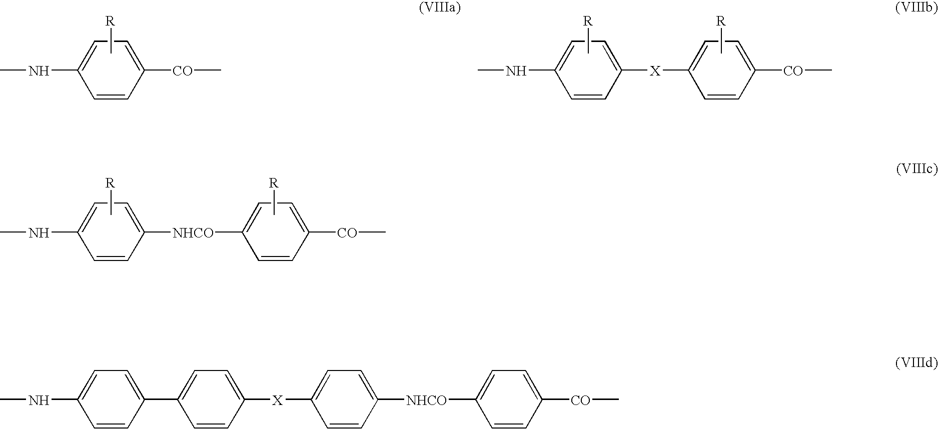
aromatic hydrazides of formulae (IXa), (IXb) and (IXc): 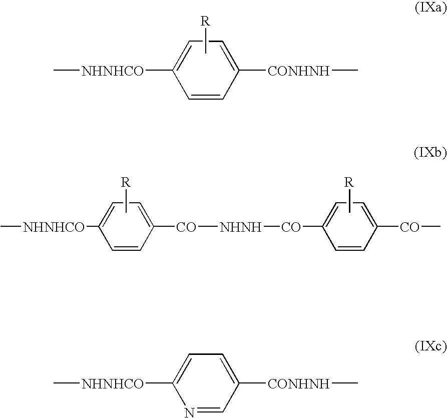
aromatic azomethines of formulae (Xa), (Xb) and (Xc): 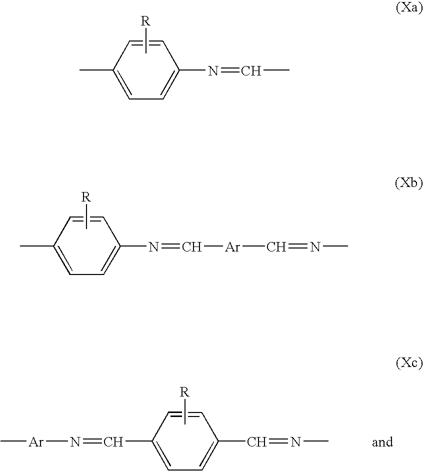
aromatic esters of formulae (XIa). (XIb) and (XIc): 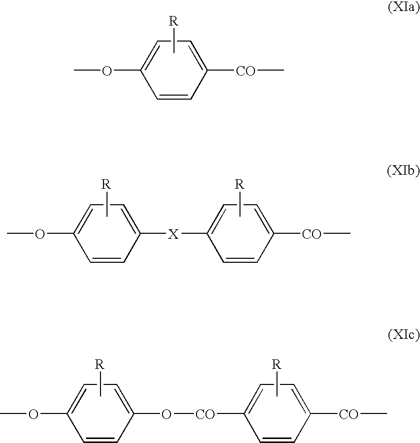
wherein in formulae (I) to (XI):
the radicals R, R1, R2, R3, and R4, which may be identical or different, are chosen from hydrogen atoms, and -R′, —OR′, —COOR′, and —OCOR′ radicals, wherein R′ is chosen from linear and branched C₁-C₂₀ alkyl radicals, halogens, nitro radicals, cyano radicals, cyanoalkyl radicals, solubilizing groups and solubilizing groups comprising a spacer group that bonds to the ring;
Ar is a radical comprising a monoaromatic or polyaromatic radical;
X is chosen from oxygen and sulfur atoms, and —NHCO—, —SO₂—, —N═N—, —C(CH₃)₂—, —CH₂—, —CH═CH—, and —CH═N— radicals;
Z is chosen from —CH═CH— and —C≡C— radicals.

37. The process according to claim 36, wherein the solubilizing groups are chosen from:
—COOH and —COO—M⁺, wherein M is chosen from alkali metals, alkaline-earth metals, organic amines, alkanolamines and amino acids,
—SO₃H and —SO₃⁻M⁺, wherein M is chosen from alkali metals, alkaline-earth metals, organic amines, alkanolamines and amino acids,
primary, secondary and tertiary amine radicals,
quaternary ammonium radicals,
hydroxyl radicals, and
poly((C₂-C₃)alkylene oxide) radicals.

38. The process according to claim 36, wherein the solubilizing groups are connected to the ring via a spacer group.

39. The process according to claim 38, wherein the spacer group is chosen from -R″-, —OR″-, —OCOR″- and —COOR″- radicals, wherein R″ is chosen from linear and branched C₁-C₂₀ alkyl radicals optionally comprising at least one hetero atom.

40. The process according to claim 38, wherein the quaternary ammonium radicals are —NR′₃⁺Z⁻ wherein Z is chosen from Br and Cl atoms, and (C₁-C₄)alkyl-OSO₃ radicals, and wherein R′, which may be identical or different, are chosen from linear and branched C₁ to C₂₀ alkyl radicals, or two R′ radicals form a heterocycle with the nitrogen.

41. A method for cosmetically treating keratin fibers comprising applying to the fibers, either wet or dry, a composition comprising, in a cosmetically acceptable medium, at least one conductive polymer and at least one direct dye, wherein the at least one conductive polymer and the at least one direct dye are present in an amount sufficient to give the fibers an optical effect.

42. The method according to claim 41, wherein the optical effect is sheen.

43. A kit comprising
at least one first compartment comprising a composition comprising, in a cosmetically acceptable medium, at least one direct dye and at least one conductive polymer, and, optionally,
at least one second compartment comprising at least one oxidizing composition comprising at least one oxidizing agent.
